(19) United States
(12) Patent Application Publication
(10) Pub. No.: US 2024/0325139 A1
(43) Pub. Date: Oct. 3, 2024

(54) INTRAOCULAR LENSES WITH HAPTIC ISOLATING STRUCTURES

(71) Applicant: Alcon Inc., Fribourg (CH)

(72) Inventors: Andrew R. WALZ, Longmont, CO (US); Gregory Vinton MATTHEWS, San Francisco, CA (US); Stephen COLLINS, San Francisco, CA (US); Raza Ulabdin SHAH, San Francisco, CA (US); Hanyang HUANG, Santa Clara, CA (US); William DOLLA, Plano, TX (US); Paul MISSEL, Arlington, TX (US)

(73) Assignee: Alcon Inc., Fribourg (CH)

(21) Appl. No.: 18/616,063

(22) Filed: Mar. 25, 2024

Related U.S. Application Data
(60) Provisional application No. 63/492,435, filed on Mar. 27, 2023.
(60) Provisional application No. 63/492,430, filed on Mar. 27, 2023.

(51) Int. Cl. A61F 2/16

(52) U.S. Cl. CPC A61F 2/1601; A61F 2002/16903

(57) ABSTRACT

Disclosed herein are intraocular lenses comprising an optic portion and at least one haptic having a proximal end coupled to the optic portion and a distal end. The haptic comprising a haptic lumen extending through at least part of the haptic. The haptic can comprise one or more haptic isolators disposed within the haptic lumen. The one or more haptic isolators can be configured to counteract or reduce unintended shape changes caused by an external energy directed at the haptic.

### CROSS-REFERENCE TO RELATED APPLICATION

This application claims priority to U.S. Patent Application No. 63/492,430 filed on Mar. 27, 2023 and U.S. Patent Application No. 63/492,435 filed on Mar. 27, 2023, the contents of which are incorporated herein by reference in their entireties.

### TECHNICAL FIELD

The present disclosure relates generally to the field of intraocular lenses, and, more specifically, to adjustable intraocular lenses.

### BACKGROUND

A cataract is a condition involving the clouding over of the normally clear lens of a patient's eye. Cataracts occur as a result of aging, hereditary factors, trauma, inflammation, metabolic disorders, or exposure to radiation. Age-related cataract is the most common type of cataracts. In treating a cataract, the surgeon removes the native crystalline lens matrix from the patient's capsular bag and replaces it with an intraocular lens (IOL). Traditional IOLs provide one or more selected focal lengths that allow the patient to have distance vision. However, after cataract surgery, patients with traditional IOLs often require glasses or other corrective eyewear for certain activities since the eye can no longer undertake accommodation (or change its optical power) to maintain a clear image of an object or focus on an object as its distance varies.

Newer IOLs such as accommodating IOLs, allow the eye to regain at least some focusing ability. Accommodating IOLs (AIOLs) use forces available in the eye to change some portion of the optical system in order to refocus the eye on distant or near targets. In addition, there may be a need to adjust IOLs post-operatively or after implantation within the eye of a subject. In some instances, an implanted IOL may be adjusted using laser treatments.

However, such post-implant adjustment procedures may also present challenges to the overall functionality of the implanted IOL. Therefore, improved solutions are needed which address the above concerns. Such a solutions should also be designed with clinical considerations in mind.

### SUMMARY

Disclosed herein are intraocular lenses with haptic isolating structures. In some embodiments, an intraocular lens is disclosed comprising an optic portion comprising an optic fluid chamber and a haptic having a proximal end coupled to the optic portion and a distal end. The haptic can comprise a haptic lumen extending through at least part of the haptic and in fluid communication with the optic fluid chamber. The haptic can comprise a plurality of haptic isolators disposed within the haptic lumen.

The haptic isolators can be configured to limit any radial movement of the radially-outer haptic wall to between 0 and 10 microns in response to laser light directed at the haptic.

The haptic isolators can be configured to counteract or reduce unintended shape changes caused by laser light directed at the haptic.

In some embodiments, the haptic lumen can be surrounded by a radially-outer haptic wall, a radially-inner haptic wall, an anterior haptic wall, and a posterior haptic wall. The haptic isolators can extend from the anterior haptic wall to the posterior haptic wall of the haptic.

In some embodiments, each of the haptic isolators can comprise lateral sides with none of the lateral sides of the haptic isolators physically contacting the radially-inner haptic wall or the radially-outer haptic wall.

In some embodiments, the haptic isolators can be positioned radially closer to the radially-inner haptic wall than the radially-outer haptic wall. A lateral side of at least one of the haptic isolators closest to the radially-inner haptic wall can be separated from the radially-inner haptic wall by an inner separation distance. In addition, another lateral side of the haptic isolator closest to the radially-outer haptic wall can be separated from the radially-outer haptic wall by an outer separation distance. The outer separation distance can be between 1.5× to 3× greater than the inner separation distance.

In some embodiments, at least one of the haptic isolators can be configured as a column with a substantially circular transverse cross-section.

In some embodiments, at least one of the haptic isolators can have a substantially rectangular transverse cross-section, a substantially triangular transverse cross-section, or a substantially oval transverse cross-section.

In some embodiments, the haptic isolators can be arranged as a curved colonnade within the haptic lumen.

In some embodiments, the haptic isolators can be positioned at fixed intervals along at least a segment of the haptic lumen.

In some embodiments, the haptic can comprise between three and twenty haptic isolators. In other embodiments, the haptic can comprise between twenty and thirty haptic isolators.

In some embodiments, each of the haptic isolators can comprise an isolator anterior end, an isolator posterior end, and an isolator segment in between the isolator anterior end and the isolator posterior end. A width or diameter of at least one of the isolator anterior end and the isolator posterior end can be greater than the isolator segment in between the isolator anterior end and the isolator posterior end.

In some embodiments, a width or diameter of at least one of the haptic isolators can remain constant along a length or height of the haptic isolator.

In some embodiments, each of the haptic isolators can be measurable by an isolator width or diameter and an isolator length or height. The isolator length or height of at least one of the haptic isolators can be more than double the isolator width or diameter.

In some embodiments, the haptic isolators can be arranged in the shape of an arc. The haptic isolators can comprise a distally-most haptic isolator and a proximally-most haptic isolator serving as endpoints of the arc. The arc formed by the haptic isolators can be measurable by a central angle or arc angle. The central angle or arc angle can be between 70 degrees and 74 degrees.

In some embodiments, the haptic can comprise at least one of a lumen filler and a lumen expander made of a composite material. The composite material can be configured to expand in response to receiving the laser light directed at the lumen filler or the lumen expander.

In some embodiments, the haptic isolators can be made of the same material as one or more walls of the haptic and are not made of the composite material.

Also disclosed is an intraocular lens comprising an optic portion and a haptic having a proximal end coupled to the optic portion and a distal end. The haptic can comprise a haptic lumen extending through at least part of the haptic. A plurality of haptic isolators can be disposed within the haptic lumen in an arc formation.

In some embodiments, the haptic isolators can be configured to counteract or reduce unintended shape changes caused by laser light directed at the haptic. For example, the haptic isolators can be configured to limit any radial movement of the radially-outer haptic wall to between 0 and 10 microns in response to laser light directed at the haptic.

In some embodiments, the haptic comprises at least one of a lumen filler and a lumen expander made of a composite material. The composite material can be configured to expand in response to receiving the laser light directed at the lumen filler or the lumen expander. haptic isolators are not made of the composite material.

In some embodiments, the haptic isolators can be made of the same material as one or more walls of the haptic and are not made of the composite material.

In some embodiments, the haptic lumen can be surrounded by a radially-outer haptic wall, a radially-inner haptic wall, an anterior haptic wall, and a posterior haptic wall. The haptic isolators can extend from the anterior haptic wall to the posterior haptic wall of the haptic.

In some embodiments, the haptic isolators can be positioned radially closer to the radially-inner haptic wall than the radially-outer haptic wall. A lateral side of at least one of the haptic isolators closest to the radially-inner haptic wall can be separated from the radially-inner haptic wall by an inner separation distance. In addition, another lateral side of the haptic isolator closest to the radially-outer haptic wall can be separated from the radially-outer haptic wall by an outer separation distance. The outer separation distance can be between 1.5× to 3× greater than the inner separation distance.

In some embodiments, each of the haptic isolators can comprise lateral sides with none of the lateral sides of the haptic isolators physically contacting the radially-inner haptic wall or the radially-outer haptic wall.

In some embodiments, at least one of the haptic isolators can be configured as a column with a substantially circular transverse cross-section.

In some embodiments, at least one of the haptic isolators can have a substantially rectangular transverse cross-section, a substantially triangular transverse cross-section, or a substantially oval transverse cross-section.

In some embodiments, the haptic isolators can be arranged as a curved colonnade within the haptic lumen.

In some embodiments, the haptic isolators can be positioned at fixed intervals along at least a segment of the haptic lumen.

In some embodiments, the haptic can comprise between three and fourteen haptic isolators.

In some embodiments, each of the haptic isolators can comprise an isolator anterior end, an isolator posterior end, and an isolator segment in between the isolator anterior end and the isolator posterior end. A width or diameter of at least one of the isolator anterior end and the isolator posterior end can be greater than the isolator segment in between the isolator anterior end and the isolator posterior end.

In some embodiments, a width or diameter of at least one of the haptic isolators can remain constant along a length or height of the haptic isolator.

In some embodiments, each of the haptic isolators can be measurable by an isolator width or diameter and an isolator length or height. The isolator length or height of at least one of the haptic isolators can be more than double the isolator width or diameter.

In some embodiments, the haptic isolators can be arranged in the shape of an arc. The haptic isolators can comprise a distally-most haptic isolator and a proximally-most haptic isolator serving as endpoints of the arc. The arc formed by the haptic isolators can be measurable by a central angle or arc angle. The central angle or arc angle can be between 70 degrees and 74 degrees.

Also disclosed is an intraocular lens comprising: an optic portion and a haptic having a proximal end coupled to the optic portion and a distal end. The haptic can comprise a haptic lumen extending through at least part of the haptic. The haptic can comprise one or more isolating blocks disposed within the haptic lumen. At least one of the one or more isolating blocks can have a non-circular transverse cross-section.

In some embodiments, the one or more isolating blocks can be configured to counteract or reduce unintended shape changes caused by laser light directed at the haptic. For example, the one or more isolating blocks can be configured to limit any radial movement of the radially-outer haptic wall to between 0 and 10 microns in response to laser light directed at the haptic.

In some embodiments, the haptic can comprise at least one of a lumen filler and a lumen expander made of a composite material. The composite material can be configured to expand in response to receiving the laser light directed at the lumen filler or the lumen expander.

In some embodiments, the one or more isolating blocks can be made of the same material as one or more walls of the haptic and are not made of the composite material.

In some embodiments, the haptic lumen can be surrounded by a radially-outer haptic wall, a radially-inner haptic wall, an anterior haptic wall, and a posterior haptic wall. The one or more isolating blocks can extend from the anterior haptic wall to the posterior haptic wall of the haptic.

In some embodiments, each of the one or more isolating blocks comprises lateral sides and none of the lateral sides of the one or more isolating blocks physically contact the radially-inner haptic wall or the radially-outer haptic wall.

In some embodiments, the one or more isolating blocks can be positioned radially closer to the radially-inner haptic wall than the radially-outer haptic wall.

In some embodiments, a lateral side of at least one of the isolating blocks closest to the radially-inner haptic wall can be separated from the radially-inner haptic wall by an inner separation distance. Another lateral side of the isolating block closest to the radially-outer haptic wall can be separated from the radially-outer haptic wall by an outer separation distance. The outer separation distance can be between 1.5× to 3× greater than the inner separation distance.

In some embodiments, at least one of the isolating blocks can have a substantially obround-shaped transverse cross-section.

In some embodiments, at least one of the isolating blocks can have a substantially rectangular-shaped transverse cross-section.

In some embodiments, at least one of the isolating blocks can have a substantially oval-shaped transverse cross-section.

In some embodiments, the haptic can comprise a plurality of isolating blocks disposed within the haptic lumen. The isolating blocks can be positioned at fixed intervals along at least a segment of the haptic lumen.

### BRIEF DESCRIPTION OF THE DRAWINGS

FIG. 1A illustrates a top plan view of one embodiment of an IOL comprising haptics with haptic isolators.

FIGS. 1B and 1C illustrate cross-sectional views of the IOL of FIG. 1A taken along cross-section A-A.

FIG. 2A illustrates part of a haptic of an IOL without haptic isolators.

FIG. 2B is a graph showing the movement of a radially-outer haptic wall of a haptic without haptic isolators and the movement of a radially-outer haptic wall of a haptic with haptic isolators

FIG. 3 illustrates a cross-sectional view of a haptic comprising haptic isolators.

FIG. 4A illustrates a perspective view of one embodiment of a haptic comprising haptic isolators with an anterior portion of the haptic removed for ease of viewing.

FIG. 4B illustrates a top plan view of another embodiment of a haptic comprising haptic isolators.

FIG. 5A illustrates a perspective view of a haptic comprising haptic isolators.

FIG. 5B illustrates the haptic of FIG. 5A with a distal end of the haptic removed to show a cross-section of part of the haptic.

FIG. 6 is a computed tomography (CT) scan image of part of a haptic comprising haptic isolators with a radially outer wall of the haptic digitally removed.

FIG. 7A illustrates a perspective view of another embodiment of a haptic comprising a different type of haptic isolators with an anterior portion of the haptic removed for ease of viewing.

FIG. 7B illustrates a perspective view of yet another embodiment of a haptic comprising another type of haptic isolators with an anterior portion of the haptic removed for ease of viewing.

### DETAILED DESCRIPTION

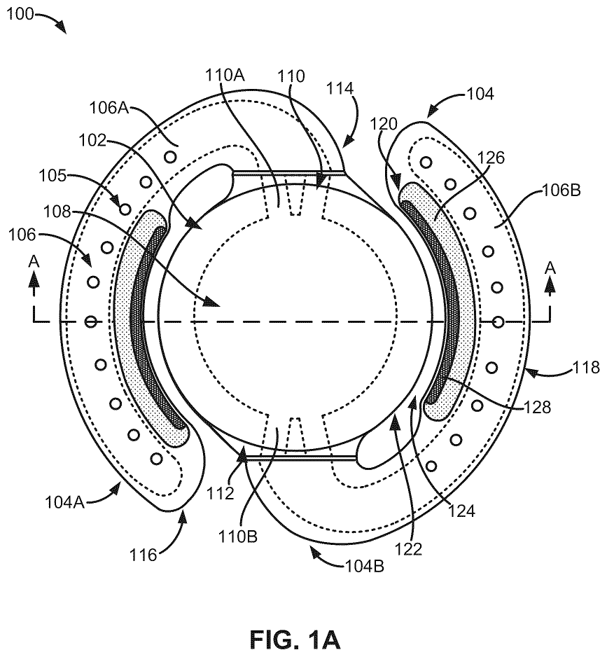

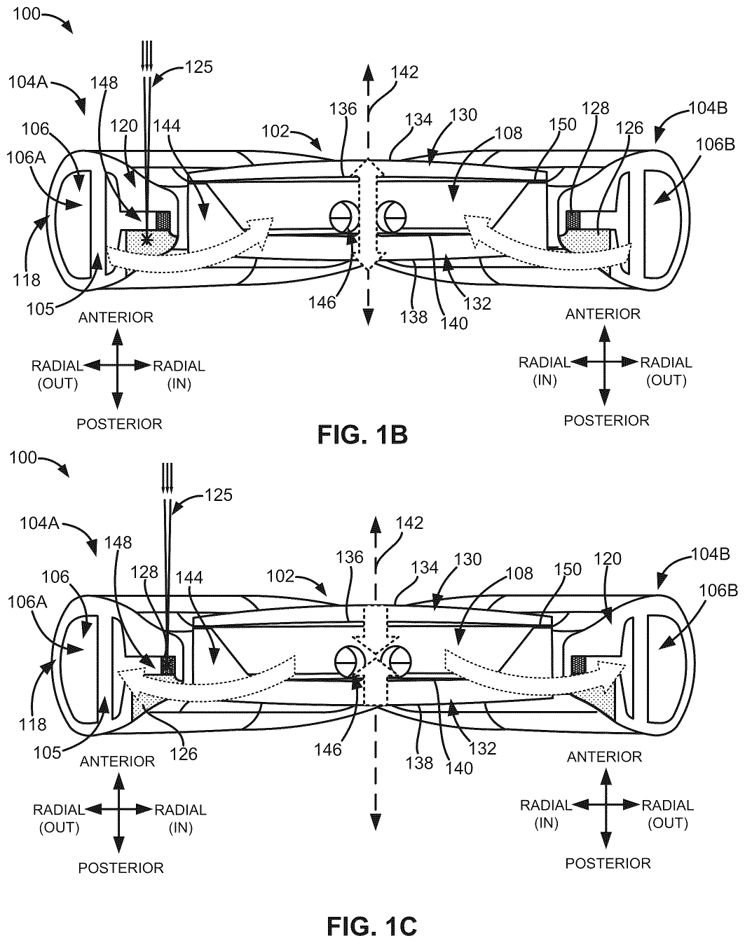

FIG. 1A illustrates a top plan view of one embodiment of an IOL 100 comprising haptics 104 with haptic isolators 105. In some embodiments, the IOL 100 can be an adjustable IOL such as an accommodating IOL (AIOL). The IOL 100 can be implanted within a subject to correct for defocus aberration, corneal astigmatism, spherical aberration, or a combination thereof.

The IOL 100 can comprise an optic portion 102 and one or more haptics 104 comprising a first haptic 104A and a second haptic 104B coupled to and extending peripherally from the optic portion 102. The IOL 100 can be positioned within a native capsular bag in which a native lens has been removed.

When implanted within the native capsular bag, the optic portion 102 can be adapted to refract light that enters the eye onto the retina. The one or more haptics 104 can be configured to engage the capsular bag and be adapted to deform in response to ciliary muscle movement (e.g., muscle relaxation, muscle contraction, or a combination thereof) in connection with capsular bag reshaping.

Each of the haptics 104 can comprise a haptic lumen 106 extending through at least part of the haptic 104. For example, the first haptic 104A can comprise a first haptic lumen 106A extending through at least part of the first haptic 104A and the second haptic 104B can comprise a second haptic lumen 106B extending through at least part of the second haptic 104B. The haptic lumen 106 (e.g., any of the first haptic lumen 106A or the second haptic lumen 106B) can be in fluid communication with or fluidly connected to an optic fluid chamber 108 within the optic portion 102.

The optic fluid chamber 108 can be in fluid communication with the one or more haptic lumens 106 through one or more fluid channels 110. The fluid channels 110 can be conduits or passageways fluidly connecting the optic fluid chamber 108 to the haptic lumens 106. The fluid channels 110 can be spaced apart from one another. For example, a pair of fluid channels 110 can be spaced apart between about 0.1 mm to about 1.0 mm. In some embodiments, each of the fluid channels 110 can have a diameter of between about 0.4 mm to about 0.6 mm.

The haptics 104 can be coupled to the optic portion 102 at a reinforced portion 112. The reinforced portion 112 can serve as a haptic-optic interface. The pair of fluid channels 110 can be defined or formed within part of the reinforced portion 112.

As shown in FIG. 1A, the optic fluid chamber 108 can be in fluid communication with the first haptic lumen 106A through a first pair of fluid channels 110A. The optic fluid chamber 108 can also be in fluid communication with the second haptic lumen 106B through a second pair of fluid channels 1101B.

In some embodiments, the first pair of fluid channels 110A and the second pair of fluid channels 110B can be positioned substantially on opposite sides of the optic portion 102. The first pair of fluid channels 110A can be positioned substantially diametrically opposed to the second pair of fluid channels 110B. The first pair of fluid channels 110A and the second pair of fluid channels 110B can be defined or extend through part of the optic portion 102. The first pair of fluid channels 110A and the second pair of fluid channels 110B can be defined or extend through a posterior element 132 of the optic portion 102 (see, e.g., FIGS. 1B and 1C).

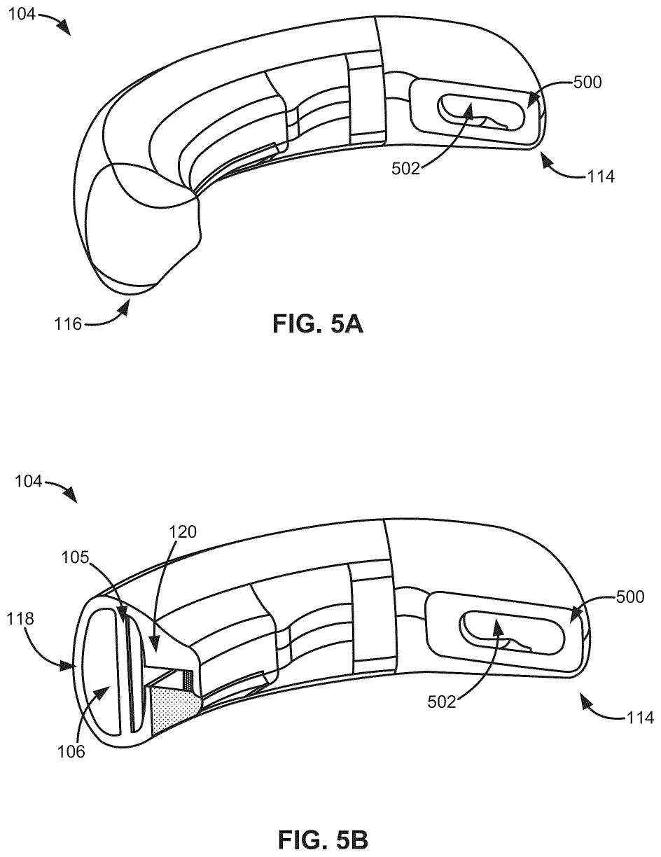

FIG. 1A also illustrates that each of the haptics 104 (e.g., any of the first haptic 104A or the second haptic 104B) can have a proximal attachment end 114 and a distal free end 116. A haptic fluid port 502 (see, e.g., FIGS. 5A and 5B) can be defined at the proximal attachment end 114 of the haptic 104. The haptic fluid port 502 can serve as an opening of the haptic lumen 106. Fluid within the haptic lumen 106 can flow out of the haptic lumen 106 through the haptic fluid port 502 and into the optic fluid chamber 108 via the fluid channels 110 when the haptic 104 is coupled to the optic portion 102. Similarly, fluid within the optic fluid chamber 108 can flow out of the optic fluid chamber 108 through the pair of fluid channels 110 and into the haptic lumen 106 through the haptic fluid port 502.

Each of the haptics 104 can comprise a radially-outer haptic wall 118 and a radially-inner haptic wall 120. The radially-outer haptic wall 118 can be configured to face and contact an inner surface of a patient's capsular bag when the IOL 100 is implanted within the capsular bag. The radially-inner haptic wall 120 can be configured to face an outer peripheral surface 112 of the optic portion 102.

The IOL 100 can be implanted or introduced into a patient's capsular bag after a native lens has been removed from the capsular bag. The patient's capsular bag is connected to zonule fibers which are connected to the patient's ciliary muscles. The capsular bag is elastic and ciliary muscle movements can reshape the capsular bag via the zonule fibers. For example, when the ciliary muscles relax, the zonules are stretched. This stretching pulls the capsular bag in the generally radially outward direction due to radially outward forces. This pulling of the capsular bag causes the capsular bag to elongate, creating room within the capsular bag. When the patient's native lens is present in the capsular bag, the native lens normally becomes flatter (in the anterior-to-posterior direction), which reduces the power of the lens, allowing for distance vision. In this configuration, the patient's native lens is said to be in a disaccommodated state or undergoing disaccommodation.

When the ciliary muscles contract, however, as occurs when the eye is attempting to focus on near objects, the radially inner portion of the muscles move radially inward, causing the zonules to slacken. The slack in the zonules allows the elastic capsular bag to contract and exert radially inward forces on a lens within the capsular bag. When the patient's native lens is present in the capsular bag, the native lens normally becomes more curved (e.g., the anterior part of the lens becomes more curved), which gives the lens more power, allowing the eye to focus on near objects. In this configuration, the patient's native lens is said to be in an accommodated state or undergoing accommodation.

When the IOL 100 is implanted into a patient's capsular bag, the radially-outer haptic wall 118 of the haptics 104 can directly engage with or be in physical contact with the portion of the capsular bag that is connected to the zonules or zonule fibers. Therefore, the radially-outer haptic wall 118 can be configured to respond to capsular bag reshaping forces that are applied radially when the zonules relax and stretch as a result of ciliary muscle movements.

When the ciliary muscles contract, the peripheral region of the elastic capsular bag reshapes and applies radially inward forces on the radially-outer haptic wall 118 of each of the haptics 104. The radially-outer haptic wall 118 can then deform or otherwise changes shape and this deformation or shape-change can cause the volume of the haptic lumen 106 to decrease. When the volume of the haptic lumen 106 decreases, the fluid within the haptic lumen 106 is moved or pushed into the optic fluid chamber 108.

The optic portion 102 can change shape in response to fluid entering the optic fluid chamber 108 from the haptic lumen 106. This can increase the base power or base spherical power of the IOL 100 and allow a patient with the IOL 100 implanted within the eye of the patient to focus on near objects. In this state, the IOL 100 can be considered to have undergone accommodation.

When the ciliary muscles relax, the peripheral region of the elastic capsular bag is stretched radially outward and the capsular bag elongates. The radially-outer haptic wall 118 of the haptics 104 can be configured to respond to this capsular bag reshaping by returning to its non-deformed or non-stressed configuration. This causes the volume of the haptic lumen 106 to increase or return to its non-deformed volume. This increase in the volume of the haptic lumen 106 can cause the fluid within the optic fluid chamber 108 to be drawn out or otherwise flow out of the optic fluid chamber 108 and back into the haptic lumen 106. As discussed previously, fluid moves out of the optic fluid chamber 108 into the haptic lumen 106 through the same fluid channels 110 formed within the optic portion 102.

As previously discussed, the optic portion 102 can change shape in response to fluid exiting the optic fluid chamber 108 and into the haptic lumen 106. This can decrease the base power or base spherical power of the IOL 100 and allow a patient with the IOL 100 implanted within the eye of the patient to focus on distant objects or provide for distance vision. In this state, the IOL 100 can be considered to have undergone disaccommodation.

In some embodiments, the IOL 100 can be designed such that a gap 124 or void space radially separates the radially-inner haptic wall 120 of the haptic 104 from the outer peripheral surface 122 of the optic portion 102. This can allow portions of the haptic 104 to change shape or expand in response to an external energy (e.g., laser energy) directed at the haptic 104.

FIG. 1A also illustrates that one or more portions of each of the haptics 104 can be made of a composite material. As will be discussed in more detail in later sections, the composite material can comprise or be made in part of an energy absorbing constituent, a plurality of expandable components, and a cross-linked copolymer used to make the rest of the haptic 104. The portions of the haptics 104 made of the composite material can be configured to change shape (e.g., expand) in response to the laser light 125 (see, e.g., FIGS. 1B-1C) directed at the composite material. Depending on where the composite material is positioned or integrated within each of the haptics 104, the composite material can act as a lumen filler to take up space within the haptic lumen 106 and/or a lumen expander 128 to create more space within the haptic lumen 106.

As will be discussed in more detail in later sections, when laser light 125 is applied to the composite material configured as the lumen filler 126, the composite material can expand and the expansion of the composite material in this instance can decrease a volume of the haptic lumen 106 and cause fluid within the haptic lumen 106 to be displaced into the optic fluid chamber 108. This can cause the optic portion 102 to change shape (e.g., cause the anterior or posterior elements of the optic portion 102 to become more curved) leading to an increase in the base power of the optic portion 102.

Alternatively, when the laser light 125 is applied to the composite material configured as the lumen expander 128, the composite material can expand and the expansion of the composite material in this instance can increase a volume of the haptic lumen 106 and cause fluid within the optic fluid chamber 108 to be drawn into the haptic lumen 106. This can also cause the optic portion 102 to change shape (e.g., cause the anterior or posterior elements of the optic portion 102 to become less curved or flatter) leading to a decrease in the base power of the optic portion 102.

As will be discussed in more detail in the ensuing sections, each of the haptics 104 can comprise a plurality of haptic isolators 105 that can counteract or reduce the effects of unintended shape changes or deformations caused by the application of laser light 125 to the haptics 104.

Although AIOLs are depicted and described in this disclosure, any reference to an AIOL can also refer to one of the AIOLs discussed and depicted in the following U.S. publications: U.S. Pat. Pub. No. 2021/0100652; U.S. Pat. Pub. No. 2021/0100650; U.S. Pat. Pub. No. 2020/0337833; and U.S. Pat. Pub. No. 2018/0153682; and in the following issued U.S. patents: U.S. Pat. Nos. 11,426,270; 10,433,949; 10,299,913; 10,195,020; and 8,968,396, the contents of which are incorporated herein by reference in their entireties.

FIGS. 1B and 1C illustrate cross-sectional views of the IOL 100 of FIG. 1A taken along cross-section A-A. As shown in FIGS. 1B and 1C, the optic portion 102 can comprise an anterior element 130 and a posterior element 132. The fluid-filled optic fluid chamber 108 can be defined in between the anterior element 130 and the posterior element 132.

The anterior element 130 can comprise an anterior optical surface 134 and an anterior inner surface 136 opposite the anterior optical surface 134. The posterior element 132 can comprise a posterior optical surface 138 and a posterior inner surface 140 opposite the posterior optical surface 138. Any of the anterior optical surface 134, the posterior optical surface 138, or a combination thereof can be considered and referred to as an external optical surface. The anterior inner surface 136 and the posterior inner surface 140 can face the optic fluid chamber 108. At least part of the anterior inner surface 136 and at least part of the posterior inner surface 140 can serve as chamber walls of the optic fluid chamber 108.

As shown in FIGS. 1B and 1C, the optic portion 102 can have a lens optical axis 142 extending in an anterior-to-posterior direction through a center of the optic portion 102. The lens optical axis 142 can extend through the centers of both the anterior element 130 and the posterior element 132.

The thickness of the anterior element 130 can be greater at or near the lens optical axis 142 than at the periphery of the anterior element 130. In some embodiments, the thickness of the anterior element 130 can increase gradually from the periphery of the anterior element 130 toward the lens optical axis 142.

In certain embodiments, the thickness of the anterior element 130 at or near the lens optical axis 142 can be between about 0.45 mm and about 0.55 mm. In these and other embodiments, the thickness of the anterior element 130 near the periphery can be between about 0.20 mm and about 0.40 mm. Moreover, the anterior inner surface 136 of the anterior element 130 can have less curvature or be flatter than the anterior optical surface 134.

The thickness of the posterior element 132 can be greater at or near the lens optical axis 142 than portions of the posterior element 132 radially outward from the lens optical axis 142 but prior to reaching a raised periphery 144 of the posterior element 132. The thickness of the posterior element 132 can gradually decrease from the lens optical axis 142 to portions radially outward from the lens optical axis 142 (but prior to reaching the raised periphery 144). As shown in FIGS. 1B and 1C, the thickness of the posterior element 132 can increase once again from a radially inner portion of the raised periphery 144 to a radially outer portion of the raised periphery 144.

In certain embodiments, the thickness of the posterior element 132 at or near the lens optical axis 142 can be between about 0.45 mm and about 0.55 mm. In these and other embodiments, the thickness of the posterior element 132 radially outward from the lens optical axis 142 (but prior to reaching the raised periphery 144) can be between about 0.20 mm and about 0.40 mm. The thickness of the posterior element 132 near the radially outer portion of the raised periphery 144 can be between about 1.00 mm and 1.15 mm. Moreover, the posterior inner surface 140 of the posterior element 132 can have less curvature or be flatter than the posterior optical surface 138.

The optic portion 102 can have a base power or base spherical power. The base power of the optic portion 102 can be configured to change based on an internal fluid pressure within the fluid-filled optic fluid chamber 108. The base power of the optic portion 102 can be configured to increase or decrease as fluid enters or exits the fluid-filled optic fluid chamber 108.

The base power of the optic portion 102 can be configured to increase as fluid enters the fluid-filled optic fluid chamber 108 from the haptic lumen(s) 106, as depicted in FIG. 1B using the curved broken-line arrows. For example, the anterior element 130 of the optic portion 102 can be configured to increase its curvature in response to the fluid entering the optic fluid chamber 108. Also, for example, the posterior element 132 of the optic portion 102 can be configured to increase its curvature in response to the fluid entering the optic fluid chamber 108. In further embodiments, both the anterior element 130 and the posterior element 132 can be configured to increase their curvatures in response to the fluid entering the optic fluid chamber 108.

The base power of the optic portion 102 can be configured to decrease as fluid exits or is drawn out of the fluid-filled optic fluid chamber 108 into the haptic lumen(s) 106, as depicted in FIG. 1C using the curved broken-line arrows. For example, the anterior element 130 of the optic portion 102 can be configured to decrease its curvature (or flatten out) in response to the fluid exiting the optic fluid chamber 108. Also, for example, the posterior element 132 of the optic portion 102 can be configured to decrease its curvature (or flatten out) in response to the fluid exiting the optic fluid chamber 108. In further embodiments, both the anterior element 130 and the posterior element 132 can be configured to decrease their curvatures in response to the fluid exiting the optic fluid chamber 108.

It should be noted that although FIGS. 1B and 1C illustrate fluid entering and exiting the optic fluid chamber 108 from the haptic lumens 106 using the curved broken-line arrows, fluid enters and exits the optic fluid chamber 108 via the fluid channels 110 and apertures 146 defined along the posterior element 132. The apertures 146 can be holes or openings defined along the posterior element 132 that serve as terminal ends of the fluid channels 110. When the IOL 100 comprises a pair of fluid channels 110, the pair of apertures 146 serving as ends of the fluid channels 110 can be spaced apart from one another between about 0.1 mm to about 1.0 mm.

As shown in FIGS. 1B and 1C, one or more portions of the IOL 100 can be made of a composite material designed to respond to an external energy, such as laser light 125, applied to the composite material. For example, one or more portions of each of the haptics 104 of the IOL 100 can be made of the composite material.

In some embodiments, the laser light 125 can be a green laser light with a wavelength between about 480 nm and 650 nm (e.g., 532 nm). In these embodiments, the laser generating the laser light 125 can be a neodymium-doped yttrium aluminum garnet (Nd:YAG).

In other embodiments, the laser light 125 can have a wavelength between 1030 nm and 1035 nm. In these embodiments, the laser generating the laser light 125 can be a femtosecond laser.

Depending on where the composite material is positioned or integrated within each of the haptics 104 and the composition of the composite material, the composite material can act as a lumen filler 126 or a lumen expander 128.

For example, the lumen filler 126 can be a portion of the haptic 104 made of the composite material that is designed to decrease a volume of the haptic lumen 106 in response to an external energy (e.g., laser light 125) directed at the lumen filler 126. The lumen expander 128 can be a portion of the haptic 104 made of the composite material that is designed to increase a volume of the haptic lumen 106 in response to an external energy (e.g., laser light 125) directed at the lumen expander 128.

As shown in FIGS. 1B and 1C, each of the haptics 104 can comprise a channel 148. The channel 148 can be defined within part of the radially-inner haptic wall 120. For example, the channel 148 can extend partially into the radially-inner haptic wall 120. The channel 148 can be in fluid communication with the haptic lumen 106 or be considered part of the haptic lumen 106.

In some embodiments, the lumen filler 126 can be positioned posterior to the channel 148. In these embodiments, the lumen filler 126 can replace or act as the posterior portion of the radially-inner haptic wall 120. The lumen filler 126 can also be positioned radially inward of the portion of the haptic lumen 106 that is not the channel 148.

At least part of the lumen filler 126 can be in fluid communication with the channel 148. For example, at least part of an anterior portion or layer of the lumen filler 126 can be in fluid communication with or otherwise exposed to the channel 148.

As shown in FIGS. 1B and 1C, in some embodiments, a radially outer lateral side of the lumen filler 126 is not in fluid communication with the haptic lumen 106. In these embodiments, the radially outer lateral side of the lumen filler 126 is separated from the haptic lumen 106 by a part of the haptic 104 not made of the composite material.

The lumen expander 128 can be positioned radially inward of the channel 148. The lumen expander 128 can also be positioned anterior to the lumen filler 126. More specifically, for example, the lumen expander 128 can be positioned anterior to a radially inner portion of the lumen filler 126.

In some embodiments, the lumen expander 128 can be positioned within the channel 148. In these embodiments, the lumen expander 128 can be positioned at a radially innermost end of the channel 148. For example, the radially-inner haptic wall 120 can taper in shape as the radially-inner haptic wall 120 gets closer to the optic portion 102. The lumen expander 128 can be positioned at a radially innermost end of the channel 148 near the tapered end of the radially-inner haptic wall 120.

As shown in FIGS. 1B and 1C, a radially outer lateral side of the lumen expander 128 can be in fluid communication with the channel 148 and the haptic lumen 106. In some embodiments, the lumen expander 128 does not extend all the way to the radially inner-most part of the radially-inner haptic wall 120. In these embodiments, a part of the haptic 104 that is not made of the composite material can serve as the radially inner-most part of the radially-inner haptic wall 120 and separate the lumen expander 128 from the outer peripheral surface 122 of the optic portion 102.

In some embodiments, the lumen expander 128 can be connected or otherwise coupled to the lumen filler 126. In these and other embodiments, the lumen expander 128 and the lumen filler 126 can be or refer to different parts of the same composite material. For example, the lumen filler 126 can be shaped substantially as a curved cornice and the lumen expander 128 can be shaped substantially as a rectangular cuboid extending from an anterior surface of the cornice.

It should be understood by one of ordinary skill in the art that even though different colored shading is used to differentiate the lumen filler 126 from the lumen expander 128 in the figures (that is, a darker shading pattern is used to depict the lumen expander 128 and a lighter shading pattern is used to depict the lumen filler 126), both the lumen filler 126 and the lumen expander 128 can be made of the same composite material or refer to different parts/features of the same block of composite material.

In other embodiments, the lumen filler 126 and the lumen expander 128 can be made of different types of composite materials. In these embodiments, the lumen filler 126 can be made of a first type of composite material and the lumen expander 128 can be made of a second type of composite material. In certain embodiments, the lumen filler 126 and the lumen expander 128 can be made of different colored composite materials. For example, the composite material can comprise an energy absorbing constituent such as an energy absorbing pigment or dye.

As a more specific example, either the lumen filler 126 or the lumen expander 128 can be made of a composite material comprising a black-colored energy absorbing pigment such as graphitized carbon black. In this example, if one of the lumen filler 126 or the lumen expander 128 is made of a composite material comprising graphitized carbon black, the other can be made of another type of composite material comprising a red-colored energy absorbing pigment such as an azo dye (e.g., Disperse Red 1 dye).

As shown in FIG. 1B, an external energy such as laser light 125 can be directed at the lumen filler 126 to cause at least part of the lumen filler 126 to expand and grow in size. For example, this expansion can manifest itself as a protuberance growing or jutting out of the lumen filler 126. For example, when laser light 125 is directed at the anterior portion or layer of the lumen filler 126 in fluid communication with or otherwise exposed to the channel 148, a protuberance can grow out of the anterior portion and into the channel 148. Since the channel 148 is in fluid communication with the haptic lumen 106 (or is considered part of the haptic lumen 106), the volume of the haptic lumen 106 can decrease in response to the formation of the protuberance. This can cause fluid within the haptic lumen 106 to be pushed or otherwise displaced into the optic fluid chamber 108. As a result, at least one of the anterior element 130 and the posterior element 132 can increase its curvature and the base power of the optic portion 102 can increase in response to the laser stimulus directed at the lumen filler 126.

An external energy such as the laser light 125 (e.g., laser pulses) can be directed at the lumen expander 128 to cause at least part of the lumen expander 128 to expand and grow in size. As will be discussed in more detail in later sections, this expansion can manifest itself as an expansion of the channel 148. For example, when laser light 125 is directed at the lumen expander 128, the lumen expander 128 can grow in size and enlarge the channel 148. Since the channel 148 is in fluid communication with the haptic lumen 106 (or is considered part of the haptic lumen 106), the volume of the haptic lumen 106 can increase in response to the growth of the lumen expander 128. This can cause fluid to be drawn out of the optic fluid chamber and into the haptic lumen 106. As a result, at least one of the anterior element 130 and the posterior element 132 can decrease its curvature and the base power of the optic portion 102 can decrease in response to the laser light 125 (e.g., laser pulses) directed at the lumen expander 128.

As will be discussed in more detail in the ensuing sections, each of the haptics 104 can comprise a plurality of haptic isolators 105 that can counteract or reduce the effects of unintended shape changes or deformations caused by the application of laser light 125 to the haptics 104.

In some embodiments, the fluid within the optic fluid chamber 108 and the haptic lumen(s) 106 can be an oil. More specifically, in certain embodiments, the fluid within the optic fluid chamber 108 and the haptic lumen(s) 106 can be a silicone oil or fluid. For example, the fluid can be a silicone oil made in part of a diphenyl siloxane. In other embodiments, the fluid can be a silicone oil made in part of a ratio of two dimethyl siloxane units to one diphenyl siloxane unit. More specifically, in some embodiments, the fluid can be a silicone oil made in part of diphenyltetramethyl cyclotrisiloxane or a copolymer of diphenyl siloxane and dimethyl siloxane. In further embodiments, the fluid can be a silicone oil comprising branched polymers.

The fluid (e.g., the silicone oil) can be index matched with a lens body material used to make the optic portion 102. When the fluid is index matched with the lens body material, the entire optic portion 102 containing the fluid can act as a single lens. For example, the fluid can be selected so that it has a refractive index of between about 1.48 and 1.53 (or between about 1.50 and 1.53). In some embodiments, the fluid (e.g., the silicone oil) can have a polydispersity index of between about 1.2 and 1.3. In other embodiments, the fluid (e.g., the silicone oil) can have a polydispersity index of between about 1.3 and 1.5. In other embodiments, the fluid (e.g., the silicone oil) can have a polydispersity index of between about 1.1 and 1.2. Other example fluids are described in U.S. Patent Publication No. 2018/0153682, which is herein incorporated by reference in its entirety.

The optic portion 102 can be made in part of a deformable or flexible material. In some embodiments, the optic portion 102 can be made in part of a deformable or flexible polymeric material. For example, the anterior element 130, the posterior element 132, or a combination thereof can be made in part of a deformable or flexible polymeric material. The one or more haptics 104 (e.g., the first haptic 104A, the second haptic 104B, or a combination thereof) can be made in part of the same deformable or flexible material as the optic portion 102. In other embodiments, the one or more haptics 104 can be made in part of different materials from the optic portion 102.

In some embodiments, the optic portion 102 can comprise or be made in part of a lens body material. The lens body material can be made in part of a cross-linked copolymer comprising a copolymer blend. The copolymer blend can comprise an alkyl acrylate or methacrylate, a fluoro-alkyl (meth)acrylate, and a phenyl-alkyl acrylate. It is contemplated by this disclosure and it should be understood by one of ordinary skill in the art that these types of acrylic cross-linked copolymers can be generally copolymers of a plurality of acrylates, methacrylates, or a combination thereof and the term “acrylate” as used herein can be understood to mean acrylates, methacrylates, or a combination thereof interchangeably unless otherwise specified. The cross-linked copolymer used to make the lens body material can comprise an alkyl acrylate in the amount of about 3% to 20% (wt %), a fluoro-alkyl acrylate in the amount of about 10% to 35% (wt %), and a phenyl-alkyl acrylate in the amount of about 50% to 80% (wt %). In some embodiments, the cross-linked copolymer can comprise or be made in part of an n-butyl acrylate as the alkyl acrylate, trifluoroethyl methacrylate as the fluoro-alkyl acrylate, and phenylethyl acrylate as the phenyl-alkyl acrylate. More specifically, the cross-linked copolymer used to make the lens body material can comprise n-butyl acrylate in the amount of about 3% to 20% (wt %) (e.g., between about 12% to 16%), trifluoroethyl methacrylate in the amount of about 10% to 35% (wt %) (e.g., between about 17% to 21%), and phenylethyl acrylate in the amount of about 50% to 80% (wt %) (e.g., between about 64% to 67%).

The final composition of the cross-linked copolymer used to make the lens body material can also comprise a cross-linker or cross-linking agent such as ethylene glycol dimethacrylate (EGDMA). For example, the final composition of the cross-linked copolymer used to make the lens body material can also comprise a cross-linker or cross-linking agent (e.g., EGDMA) in the amount of about 1.0%. The final composition of the cross-linked copolymer used to make the lens body material can also comprise an initiator or initiating agent (e.g., Perkadox 16) and a UV absorber.

The one or more haptics 104 can comprise or be made in part of a haptic material. The haptic material can comprise or be made in part of a cross-linked copolymer comprising a copolymer blend. The copolymer blend can comprise an alkyl acrylate, a fluoro-alkyl acrylate, and a phenyl-alkyl acrylate. For example, the cross-linked copolymer used to make the haptic material can comprise an alkyl acrylate in the amount of about 10% to 25% (wt %), a fluoro-alkyl acrylate in the amount of about 10% to 35% (wt %), and a phenyl-alkyl acrylate in the amount of about 50% to 80% (wt %). In some embodiments, the cross-linked copolymer used to make the haptic material can comprise n-butyl acrylate in the amount of about 10% to 25% (wt %) (e.g., between about 19% to about 23%), trifluoroethyl methacrylate in the amount of about 10% to 35% (wt %) (e.g., between about 14% to about 18%), and phenylethyl acrylate in the amount of about 50% to 80% (wt %) (e.g., between about 58% to about 62%). The final composition of the cross-linked copolymer used to make the haptic material can also comprise a cross-linker or cross-linking agent, such as EGDMA, in the amount of about 1.0%. The final composition of the cross-linked copolymer used to make the haptic material can also comprise a number of photoinitiators or photoinitiating agents (e.g., camphorquinone, 1-phenyl-1,2-propanedione, and 2-ethylhexyl-4-(dimenthylamino)benzoate).

In some embodiments, the refractive index of the lens body material can be between about 1.48 and about 1.53. In certain embodiments, the refractive index of the lens body material can be between about 1.50 and about 1.53 (e.g., about 1.5178).

The anterior element 130 can be attached or otherwise adhered to the posterior element 132 via adhesives 150 or an adhesive layer. The adhesive layer can be substantially annular-shaped. The adhesives 150 or adhesive layer can be positioned at a peripheral edge of the optic portion 102 in between the anterior element 130 and the posterior element 132. For example, the adhesives 150 can be positioned on top of the raised periphery 144 of the posterior element 132.

The adhesives 150 or adhesive layer can comprise or be made in part of a biocompatible adhesive. The adhesives 150 or adhesive layer can comprise or be made in part of a biocompatible polymeric adhesive.

The adhesives 150 or adhesive layer can comprise or be made in part of a cross-linkable polymer precursor formulation. The cross-linkable polymer precursor formulation can comprise or be made in part of a copolymer blend, a hydroxyl-functional acrylic monomer, and a photoinitiator.

The copolymer blend can comprise an alkyl acrylate (e.g., n-butyl acrylate in the amount of about 41% to about 45% (wt %)), a fluoro-alkyl acrylate (e.g., trifluoroethyl methacrylate in the amount of about 20% to about 24% (wt %)), and a phenyl-alkyl acrylate (phenylethyl acrylate in the amount of about 28% to about 32% (wt %)). The hydroxyl-functional acrylic monomer can be 2-hydroxyethyl acrylate (HEA). The photoinitiator can be used to facilitate curing of the adhesive. For example, the photoinitiator can be Darocur 4265 (a 50/50 blend of diphenyl(2,4,6-trimethylbenzoyl)phosphine oxide and 2-hydroxy2-methylpropiophenone).

In some embodiments, the same adhesives 150 used to bond the anterior element 130 to the posterior element 132 can also be used to bond or affix the one or more haptics 104 to the optic portion 102.

In some embodiments, the composite material can comprise a composite base material, an energy absorbing constituent, and a plurality of expandable components. As previously discussed, one or more portions of each of the haptics 104 can be made of the composite material.

The composite base material can be comprised of hydrophobic acrylic materials. For example, the composite base material can be comprised of phenylethyl acrylate (PEA), a phenylethyl methacrylate (PEMA), or a combination thereof.

In one example embodiment, the composite base material can comprise a methacrylate-functional or methacrylic-functional cross-linkable polymer and reactive acrylic monomer diluents including lauryl methacrylate (n-dodecyl methacrylate or SR313) and ADMA. By controlling the amount of lauryl methacrylate (SR313) to ADMA, the overall corresponding hardness (i.e., more ADMA) or softness (i.e., more SR313) of the cured composite material can be controlled. The methacrylate-functional or methacrylic-functional cross-linkable polymer can be made using the cross-linkable polymer precursor formulation.

The cross-linkable polymer precursor formulation can comprise the same copolymer blend used to make the optic portion and the haptics. The copolymer blend can comprise an alkyl acrylate or methacrylate (e.g., n-butyl acrylate), a fluoro-alkyl (meth)acrylate (e.g., trifluoroethyl methacrylate), and a phenyl-alkyl acrylate (e.g., phenylethyl acrylate). For example, the copolymer blend can comprise n-butyl acrylate in the amount of about 41% to about 45% (wt %), trifluoroethyl methacrylate in the amount of about 20% to about 24% (wt %), and phenylethyl acrylate in the amount of about 28% to about 32% (wt %). The cross-linkable polymer precursor formulation can comprise or be made in part of the copolymer blend, a hydroxyl-functional acrylic monomer (e.g., HEA), and a photoinitiator (e.g., Darocur 4265 or a 50/50 blend of diphenyl(2,4,6-trimethylbenzoyl)-phosphine oxide and 2-hydroxy2-methylpropiophenone).

The composite base material can comprise the methacrylate-functional or methacrylic-functional cross-linkable polymer (as discussed above) in the amount of about 50% to about 65% (e.g., about 55% to about 60%) (wt %), the reactive acrylic monomer diluent lauryl methacrylate (SR313) in the amount of about 32% to about 38% (e.g., about 32.70%) (wt %), the reactive acrylic monomer diluent adamantly methacrylate (ADMA) in the amount of about 5% to about 9% (e.g., about 7.30%) (wt %).

The composite material can be made in several operations. The first operation can comprise preparing an uncolored composite base material. The second operation can comprise mixing the composite base material with an energy absorbing constituent, expandable components, and initiators such as one or more photoinitiators, thermal initiators, or a combination thereof. The third operation can comprise placing the uncured composite material into a desired location within the haptics 104 (e.g., in proximity to the channel 148), and curing the composite material in place.

For example, the uncolored composite base material can be mixed with an energy absorbing constituent such as a dye (e.g., Disperse Red 1 dye) or pigment (graphitized carbon black). The energy absorbing constituent will be discussed in more detail below.

In some embodiments, the expandable components can make up about 5.0% to about 15.0% by weight of a final formulation of the composite material. More specifically, the expandable components can make up about 8.0% to about 12.0% (e.g., about 10.0%) by weight of a final formulation of the composite material. In these and other embodiments, the energy absorbing constituent can make up about 0.044% to about 0.44% (or about 0.55%) by weight of the final formulation of the composite material.

The photoinitiator can be Omnirad 2022 (bis(2,4,6-trimethylbenzoyl)phenyl-phosphineoxide/2-hydroxy-2-methyl-1-phenyl-propan-1-one). The photoinitiator can make up about 1.30% by weight of a final formulation of the composite material. In addition, the composite material can also comprise a thermal initiator. The thermal initiator can make up about 1.00% by weight of a final formulation of the composite material. In some embodiments, the thermal initiator can be a dialkyl peroxide such as Luperox® peroxide. In other embodiments, the thermal initiator can be Perkadox.

In some embodiments, the energy absorbing constituent can absorb the external energy (e.g., laser energy), convert the energy to heat, and conduct the energy to the composite base material to expand the composite base material.

In some embodiments, the expandable components can be expandable microspheres comprising an expandable thermoplastic shell and a blowing agent contained within the expandable thermoplastic shell. The microspheres can be configured to expand such that a diameter of at least one of the microspheres can increase about 2× the original diameter. In other embodiments, the microspheres can be configured to expand such that the diameter of at least one of the microspheres can increase about 4× or four times the original diameter. further embodiments, the microspheres can be configured to expand such that the diameter of at least one of the microspheres can increase between about 2× and about 4× (or about 3.5×) the original diameter. For example, the microspheres can have a diameter of about 12 μm at the outset. In response to an external energy applied or directed at the composite material or in response to energy transferred or transmitted to the microspheres, the diameter of the microspheres can increase to about 40 μm.

The volume of at least one of the microspheres can be configured to expand between about ten times (10×) to about 50 times (50×) in response to the external energy applied or directed at the composite material or in response to energy transferred or transmitted to the microspheres.

In some embodiments, the blowing agent can be an expandable fluid, such as an expandable gas. More specifically, the blowing agent can be a branched-chain hydrocarbon. For example, the blowing agent can be isopentane. In other embodiments, the blowing agent can be or comprise cyclopentane, pentane, or a mixture of cyclopentane, pentane, and isopentane.

Each of the expandable components can comprise a thermoplastic shell. A thickness of the thermoplastic shell can change as the expandable component increases in size. More specifically, the thickness of the thermoplastic shell can decrease as the expandable component increases in size. For example, when the expandable components are expandable microspheres, the thickness of the thermoplastic shell (i.e., its thickness in a radial direction) can decrease as the diameter of the expandable microsphere increases.

In some embodiments, the thermoplastic shell can be made in part of nitriles or acrylonitrile copolymers. For example, the thermoplastic shell can be made in part of acrylonitrile, styrene, butadiene, methyl acrylate, or a combination thereof.

As previously discussed, the expandable components can make up between about 8.0% to about 12% by weight of a final formulation of the composite material. The expandable components can make up about 10% by weight of a final formulation of the composite material.

The expandable components can be dispersed or otherwise distributed within the composite base material making up the bulk of the composite material. The composite base material can serve as a matrix for holding or carrying the expandable components. The composite material can expand in response to an expansion of the expandable components (e.g., the thermoplastic microspheres). For example, a volume of the composite material can increase in response to the expansion of the expandable components.

The composite material also comprises an energy absorbing constituent. In some embodiments, the energy absorbing constituent can be an energy absorbing colorant.

In certain embodiments, the energy absorbing colorant can be an energy absorbing dye. For example, the energy absorbing dye can be an azo dye. In some embodiments, the azo dye can be a red azo dye such as Disperse Red 1 dye. In other embodiments, the azo dye can be an orange azo dye such as Disperse Orange dye (e.g., Disperse Orange 1), a yellow azo dye such as Disperse Yellow dye (e.g., Disperse Yellow 1), a blue azo dye such as Disperse Blue dye (e.g., Disperse Blue 1), or a combination thereof.

In additional embodiments, the energy absorbing colorant can be or comprise a pigment. For example, the energy absorbing colorant can be or comprise graphitized carbon black as the pigment.

Similar to the expandable components, the energy absorbing constituent can be dispersed or otherwise distributed within the composite base material making up the bulk of the composite material. The composite base material can serve as a matrix for holding or carrying the expandable components and the energy absorbing constituent.

As previously discussed, the energy absorbing constituent can make up between about 0.025% to about 1.0% (or, more specifically, about 0.045% to about 0.45%) by weight of a final formulation of the composite material.

The energy absorbing constituent (e.g., azo dye, graphitized carbon black, or a combination thereof) can absorb or capture an external energy (e.g., light energy or, more specifically, laser light) applied or directed at the composite material. The energy absorbing constituent can absorb or capture the external energy and then transform or transfer the energy into thermal energy or heat to the expandable components.

The thermoplastic shell can soften and begin to flow as thermal energy is transferred or transmitted to expandable components. The thermoplastic shell of the expandable components can then begin to thin or reduce in thickness in response to the thermal energy transferred or transmitted to the expandable components. As the thermoplastic shell begins to soften and reduce in thickness, the blowing agent within the expandable components can expand. The blowing agent can also expand in response the thermal energy or heat transferred or transmitted to the expandable components. Expansion of the blowing agents can cause the expandable components (e.g., the thermoplastic microspheres) to expand or increase in volume. This ultimately causes the composite material to expand or increase in volume.

As previously discussed, the external energy can be laser light 125 and the energy absorbing constituent can absorb or capture the laser light 125 directed at the composite material and transform or transfer the light energy into thermal energy or heat to the expandable components. The blowing agent within the expandable components can expand or become energized in response to the thermal energy or heat. The expandable components and, ultimately, the composite material can expand or increase in volume in response to this light energy directed at the composite material.

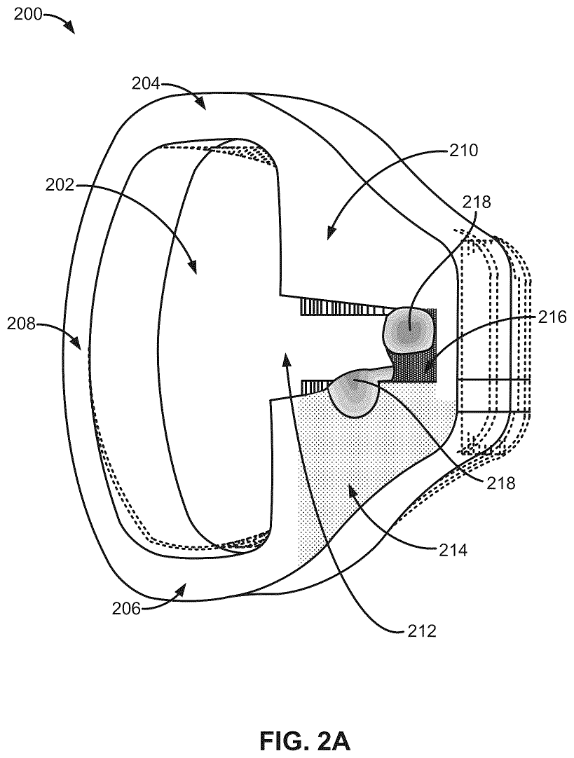

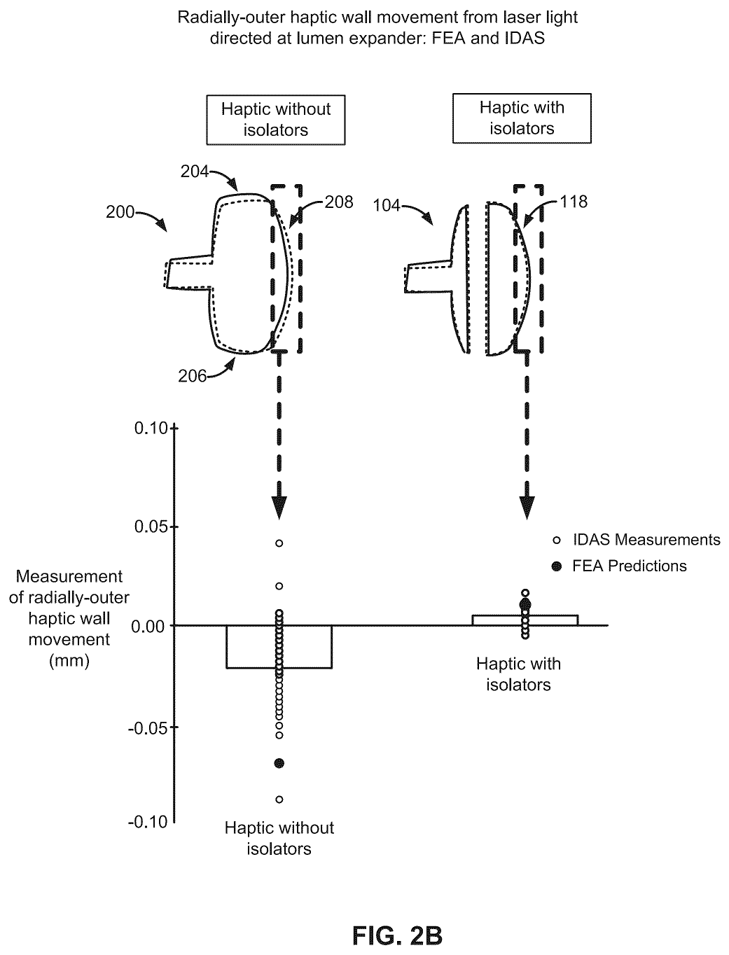

FIG. 2A illustrates part of a haptic 200 of an IOL without haptic isolators. The IOL comprising the haptic 200 shown in FIG. 2A can be similar to the IOL 100 depicted in FIGS. 1A-1C except the IOL does not have any haptic isolators 105 supporting a haptic lumen 202 of the haptic 200.

The haptic 200 can comprise an anterior haptic wall 204, a posterior haptic wall 206, a radially-outer haptic wall 208, and a radially-inner haptic wall 210 surrounding the haptic lumen 202. A lumen channel 212 can extend radially into the radially-inner haptic wall 210. Moreover, the haptic 200 can comprise a lumen filler 214, a lumen expander 216, or a combination thereof. The lumen filler 214 and the lumen expander 216 can be made of the composite material and can function similar to the lumen filler 126 and the lumen expander 128, respectively.

FIG. 2A illustrates the after-effects when laser light 125 (e.g., laser pulses from a 532 nm laser) is directed at the lumen filler 214, the lumen expander 216, or a combination thereof. For example, laser light 125 can be directed at one or more target sites 218 shown in FIG. 2A.

As shown in FIG. 2A, the composite material making up the lumen filler 214 and the lumen expander 216 can expand in response to the application of laser energy. This expansion of the composite material can also inadvertently cause other portions of the haptic 200 to change shape, expand, or bend. For example, this expansion of the composite material can inadvertently affect the size of the haptic lumen 202. Moreover, this expansion of the composite material can also cause unintended haptic bending and asymmetrical bending of the optic portion of the IOL.

The contours of the haptic 200 shown in broken lines illustrate the anterior haptic wall 204, the posterior haptic wall 206, and the radially-inner haptic wall 210 before application of laser energy. As seen in FIG. 2A, after laser light 125 is directed or otherwise delivered to the lumen filler 214, the lumen expander 216, or a combination thereof, the anterior haptic wall 204 can expand in an anterior direction and the posterior haptic wall 206 can expand in a posterior direction. In some instances, this expansion of the anterior haptic wall 204 and the posterior haptic wall 206 can cause the radially-outer haptic wall 208 to be radially pulled in or to translate in a direction of the optic portion (see FIG. 2B). These unintended shape changes can cause unwanted side effects such as unanticipated optical power changes and degradation in accommodative performance.

As such, the technical problem faced by the applicants is how to counteract or reduce the effects of these unintended haptic shape changes caused by the application of laser light 125 without interfering with the ability to fine-tune the base power of the IOL with laser-induced fluid pressure changes (i.e., without interfering with the ability to adjust the power of the lens post-implant) and without interfering with the sensitivity of the fluid-filled haptic 104 to radial forces applied by the capsular bag as a result of ciliary muscle movements (i.e., without interfering with the ability of the lens to be accommodating). The technical solution discovered and developed by the applicants are the haptic isolators 105 disclosed herein.

FIG. 2B is a graph showing measurements made of the movement of a radially-outer haptic wall 208 of a haptic 200 without haptic isolators 105 and the movement of the radially-outer haptic wall 118 of the haptic 104 with haptic isolators 105 in response to laser light 125 directed at such haptics.

FIG. 2B illustrates that measurements of the movement or displacement of the radially-outer haptic walls were made using an integrated data acquisition system (IDAS). Predictions of such movements were also made using finite element analysis (FEA) modeling. For both types of haptics, laser light 125 (not shown in FIG. 2B) was directed or otherwise delivered to a lumen filler of such haptics.

FIG. 2B shows the outer contours of the two haptics before the application of laser light using broken lines and the outer contours after the application of laser using solid lines. As can be seen in the depictions of these outer contours, the two haptics responded differently to the application of laser light 125.

In the case of the haptic 200 without haptic isolators, laser light 125 directed at the lumen expander (not shown in FIG. 2B) caused the anterior haptic wall 204 of the haptic 200 to expand in an anterior direction and the posterior haptic wall 206 of the haptic 200 to expand in a posterior direction. In the majority of cases, this expansion caused the radially-outer haptic wall 208 of the haptic 200 to be radially pulled in or to translate in a direction of the optic portion (not shown in FIG. 2B).

As can be seen in the graph of FIG. 2B, the radially-outer haptic wall 208 of the haptic 200 was displaced, on average, approximately 0.03 mm or 30 microns in a radially-inward direction (where negative values on the graph indicate radially-inward movement). In some cases, the radially-outer haptic wall 208 of the haptic 200 was measured to have moved radially-inward by up to almost 0.10 mm or 100 microns. Movement to this degree was considered problematic since it offset the desired expansion of the haptic lumen.

A different response to the laser light 125 was seen in the haptic 104 with haptic isolators 105. IDAS measurements made of such haptics 104 after the application of laser light 125 to its lumen expander (not shown in FIG. 2B) showed that the radially-outer haptic wall 208 of the haptic 104 moved to a much smaller degree.

As can be seen in the graph of FIG. 2B, the radially-outer haptic wall 118 of the haptic 104 was displaced, on average, approximately 0.01 mm or 10 microns in a radially-outward direction (where positive values on the graph indicate radially-outward movement). In some cases, the radially-outer haptic wall 118 of the haptic 104 showed no movement. In the majority of cases, the radially-outer haptic wall 118 of the haptic 104 only moved between 0 mm and 0.01 mm or 10 microns in either a radially-outward direction or a radially-inward direction.

Thus, FIG. 2B illustrates that the haptic isolators 105 can be effective at limiting any radial movement of the radially-outer haptic wall 118 in response to laser light 125 directed at the haptic 104. For example, the haptic isolators 105 can be effective at limiting any radial movement of the radially-outer haptic wall 118 to between 0 and 0.01 mm (or 10 microns) in response to laser light 125 directed at the haptic 104.

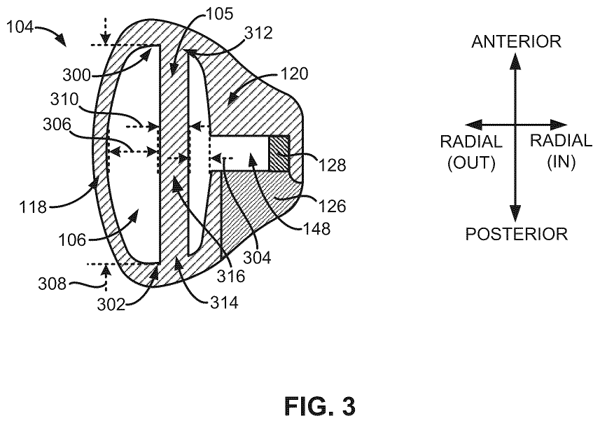

FIG. 3 illustrates a cross-sectional view of one embodiment of the haptic 104 having a haptic isolator 105 disposed within the haptic lumen 106. The haptic isolator 105 can be configured to maintain a shape of the haptic lumen 106 in response to laser energy (e.g., laser light 125) applied or delivered to the haptic 104 in order to adjust a base power of the optic portion 102.

The haptic isolator 105 can extend from an anterior haptic wall 300 to a posterior haptic wall 302. As shown in FIG. 3, the haptic isolator 105 can be aligned in an anterior-to-posterior direction such that the haptic isolator 105 is substantially parallel to the optical axis of the optic portion 102 (see, e.g., FIGS. 1B and 1C).

In other embodiments not shown in the figures but contemplated by this disclosure, the haptic isolator 105 can be tilted or slanted. For example, the haptic isolator 105 can be positioned at an angle with respect to the optical axis 142.

As shown in FIG. 3, the haptic isolator 105 can be positioned radially closer to the radially-inner haptic wall 120 than the radially-outer haptic wall 118 (see also, FIGS. 1B and 1C). This can allow the haptic isolator 105 to counteract or reduce the effects of unintended haptic shape changes caused by the application of laser light 125 without substantially affecting the sensitivity of the radially-outer haptic wall 118 to radial forces applied to the capsular bag by ciliary muscle movements.

For example, the haptic isolator 105 (or a lateral side of the haptic isolator 105 closest to the radially-inner haptic wall 120) can be separated from the radially-inner haptic wall 120 by an inner separation distance 304. The haptic isolator 105 (or another lateral side of the haptic isolator 105 closest to the radially-outer haptic wall 118) can be separated from the radially-outer haptic wall 118 by an outer separation distance 306. The outer separation distance 306 can be greater than the inner separation distance 304.

In some embodiments, the outer separation distance can be between 1.5 times (1.5×) to three times (3×) greater than the inner separation distance 304. In other embodiments, the outer separation distance can be between 1.2 times (1.2×) to 1.4 times (1.4×) greater than the inner separation distance 304. In further embodiments, the outer separation distance can be between three times (3×) to four times (4×) greater than the inner separation distance 304.

In some embodiments, the haptic isolator 105 can be positioned between the radially-inner haptic wall 120 and a centerline bisecting the haptic lumen 106 in a radial direction. For example, the haptic isolator 105 can be positioned radially inward of a centerline bisecting the haptic lumen 106 in a radial direction.

In some embodiments, the haptic isolator 105 can be cylindrical-shaped or shaped as a circular column. In other embodiments, the haptic isolator 105 can be shaped as an elongate cuboid or square prism, an elongate frusto-conic, a triangular prism, or an elongate ovoid (i.e., having an oval-shaped cross-section).

In some embodiments, the haptic isolator 105 can be made of the same material as the rest of the haptic 104. For example, the haptic isolator 105 can be made of the same cross-linked copolymer as the walls of the haptic 104. In certain embodiments, the haptic isolator 105 can be integrated with the rest of the haptic 104.

In some embodiments, the haptic isolators 105 can be formed or created during the haptic casting process. For example, the entire haptic 104, including the haptic isolators 105, can be formed by injection molding.

The haptic isolator 105 can have an isolator height 308 and an isolator width 310 or diameter (if the haptic isolator 105 is cylindrical).

In some embodiments, the isolator height 308 can be between approximately 1.5 mm to 2.00 mm. For example, the isolator height 308 can be between approximately 1.75 mm to 1.85 mm (e.g., about 1.84 mm).

In some embodiments, the isolator width 310 or diameter can be between approximately 0.20 mm to 0.60 mm. For example, the isolator width 310 can be between approximately 0.30 mm to 0.50 mm (e.g., about 0.35 mm).

In other embodiments, the isolator width 310 or diameter can be greater than 0.60 mm depending on the width of the haptic lumen 106. In further embodiments, the isolator width 310 or diameter can be less than 0.20 mm depending on the width of the haptic lumen 106.

In certain embodiments, the isolator height 308 or length can be about double the isolator width 310 or diameter. In other embodiments, the isolator height 308 or length can be more than double the isolator width 310 or diameter.

In some embodiments, a ratio of the isolator height 308 to the isolator width 310 or diameter can be between approximately 3:1 to 10:1. For example, a ratio of the isolator height to the isolator width 310 or diameter can be between approximately 4:1 to 6:1 (e.g., about 5:1).

As shown in FIG. 3, the isolator width 310 can remain constant along a length or height of the haptic isolator 105. In other embodiments, the isolator width 310 can be greater near the ends of the haptic isolator 105 (i.e., closer to the anterior haptic wall 300 and the posterior haptic wall 302) than the rest of the haptic isolator 105.

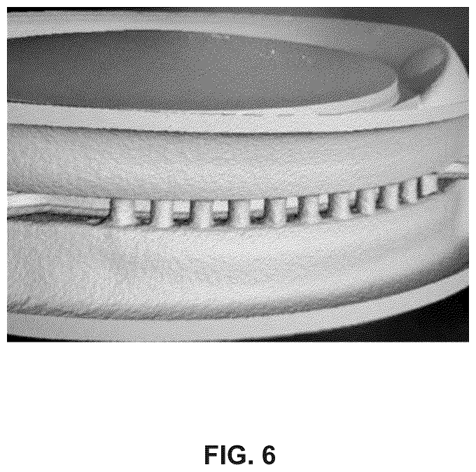

In other embodiments, each of the haptic isolators 105 can comprise an isolator anterior end 312, an isolator posterior end 314, and an isolator segment 316 in between the isolator anterior end 312 and the isolator posterior end 314. In these embodiments (see, e.g., FIG. 6) a width or diameter of at least one of the isolator anterior end 312 and the isolator posterior end 314 can be greater than the isolator segment 316 in between the isolator anterior end 312 and the isolator posterior end 314. For example, the haptic isolator 105 can widen or flare out at the isolator anterior end 312 and/or the isolator posterior end 314.

Although FIG. 3 illustrates only one haptic isolator 105 within the haptic lumen 106, it is contemplated by this disclosure that the haptic lumen 106 can comprise a plurality of haptic isolators 105 positioned along at least part of a length or segment of the haptic lumen 106.

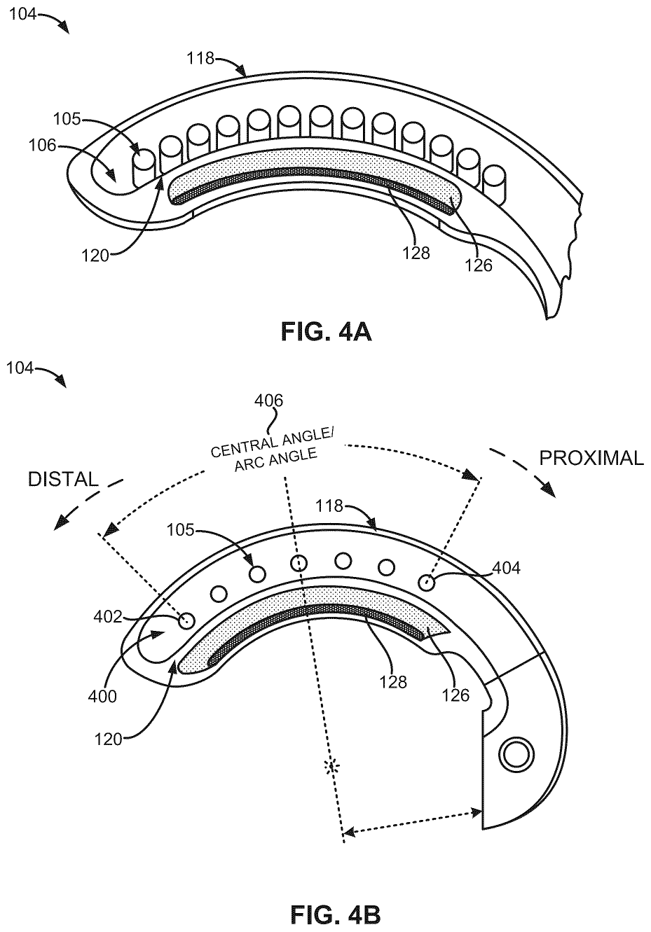

One technical problem faced by the applicants is how to counteract or reduce the effects of unintended haptic shape changes caused by the application of laser light 125. The technical solution discovered and developed by the applicants are the haptic isolators 105 disclosed herein, which are disposed within the haptic lumen 106 and are positioned radially closer to the radially-inner haptic wall 120 than the radially-outer haptic wall 118 (see also, FIGS. 1B, 1C, 4A, and 4B).

The haptic isolators 105 can act as a three-dimensional bracket or support pillar to prevent unintended deformation of the haptic lumen 106. For example, the haptic isolators 105 can prevent unintended expansion and/or contraction of the haptic lumen 106 caused by the application of laser energy to the composite material.

FIG. 4A illustrates a perspective view of one embodiment of the haptic 104 comprising a plurality of haptic isolators 105 with an anterior portion of the haptic 104 removed for ease of viewing. As previously discussed, the haptic isolators 105 can be configured to counteract or reduce any unintended shape changes caused by laser light 125 directed at the lumen filler and/or the lumen expander 128 in order to fine tune the base power of the IOL 100.

As shown in FIG. 4A, the haptic isolators 105 can be designed as columns or cylindrical pillars, each having a substantially circular transverse cross-section.

In other embodiments, at least part of the haptic isolators 105 can have a substantially triangular, rectangular, or other polygonal transverse cross-section. In further embodiments, at least part of the haptic isolators 105 can have a substantially oval transverse cross-section.

As shown in FIG. 4A, the haptic isolators 105 can be positioned within the haptic lumen 106. The haptic isolators 105 can be positioned or located such that none of the lateral sides of the haptic isolators 105 physically contact the radially-outer haptic wall 118 or the radially-inner haptic wall 120. Moreover, as shown in FIG. 4A, the haptic isolators 105 can be positioned or located radially closer to the radially-inner haptic wall 120 than the radially-outer haptic wall 118.

Moreover, as depicted in FIG. 4A, the plurality of haptic isolators 105 can be arranged as a curved colonnade within the haptic lumen 106. For example, the haptic isolators 105 can be arranged in an arc formation.

In some embodiments, the haptic isolators 105 can be positioned at fixed intervals along the length of the haptic lumen 106. In other embodiments, the haptic isolators 105 can be positioned at variable distances from one another along the length of the haptic lumen 106.

Although FIG. 4A illustrates the haptic 104 comprising thirteen haptic isolators 105, it is contemplated by this disclosure that one haptic 104 can comprise between three and twenty haptic isolators 105 (or between twenty and thirty haptic isolators 105).

The haptic isolators 105 can be made of the same haptic material used to make the haptic walls. As shown in FIG. 4A, the haptic isolators 105 are not made of the composite material used to make the lumen filler 126 and/or the lumen expander 128.

FIG. 4B illustrates a top plan view of another embodiment of a haptic 104 comprising haptic isolators 105. In the embodiment shown in FIG. 4B, the haptic 104 comprises seven haptic isolators 105. As previously discussed, one haptic 104 can comprise anywhere between three haptic isolators 105 and up to twenty haptic isolators 105 (or between twenty and thirty haptic isolators 105).

As shown in FIG. 4B, the haptic isolators 105 can be arranged in the shape of an arc 400 or be arranged in an arc-formation. The haptic isolators 105, when arranged as the arc 400, can comprise a distally-most haptic isolator 402 and a proximally-most haptic isolator 404. The arc 400 can be measurable by a central angle or arc angle 406 spanning from the distally-most haptic isolator 402 to the proximally-most haptic isolator 404. In some embodiments, this central angle or arc angle can be between 70 degrees and 74 degrees.

FIG. 5A illustrates a perspective view of one embodiment of a haptic 104 of an IOL 100 comprising haptic isolators 105. FIG. 5B illustrates the haptic 104 with a distal end 116 of the haptic 104 removed to show a cross-section of the haptic 104.

As shown in FIGS. 5A and 5B, the proximal attachment end 114 of the haptic 104 can terminate at a substantially flat interface surface 500. The flat interface surface 500 can allow the haptic 104 to adhere or otherwise couple to a corresponding interface surface (also a flat surface) protruding from the reinforced portion 112 of the optic portion 102. In some embodiments, the haptics 104 can be coupled or adhered to the optic portion 102 via the same biocompatible adhesives 150 used to adhere the anterior element 130 to the posterior element 132.

The corresponding interface surface can extend out radially from the optic portion 102. For example, the corresponding interface surface can extend out radially beyond the outer peripheral surface 122 of the optic portion 102 (e.g., extend out radially between about 10 microns and 1.0 mm beyond the outer peripheral surface 122 of the optic portion 102).

FIGS. 5A and 5B also illustrate that the substantially flat interface surface 500 can define a haptic fluid port 502. The haptic fluid port 502 can be an opening serving as a proximal end of the haptic lumen 106. When the flat interface surface 500 of the haptic 104 is coupled to the corresponding interface surface of the optic portion 102, the haptic fluid port 502 can be fluidly connected to or be in fluid communication with one or more exterior-facing apertures serving as terminal ends of the fluid channels 110. Fluid (e.g., silicone oil) entering the optic fluid chamber 108 can exit the haptic lumen 106 through the haptic fluid port 502 and into the fluid channels 110. Moreover, fluid exiting the optic fluid chamber 108 can enter the haptic lumen 106 through the haptic fluid port 502.

Although FIG. 5B illustrates only one haptic isolator 105 within the haptic lumen 106, it is contemplated by this disclosure that the haptic lumen 106 can comprise a plurality of haptic isolators 105 positioned along a length or segment of the haptic lumen 106. In some embodiments, the haptic isolators 105 can be positioned at fixed intervals along the length of the haptic lumen 106. In other embodiments, the haptic isolators 105 can be positioned at variable distances from one another along the length of the haptic lumen 106.

FIG. 6 is a computed tomography (CT) scan image of part of a haptic 104 comprising a plurality of haptic isolators 105. In this CT scan image, the radially-outer haptic wall 118 of the haptic 104 has been digitally removed for ease of viewing.

As shown in FIG. 6, the haptic isolators 105 are positioned within the haptic lumen 106 and extend in a substantially axial direction (i.e., substantially parallel to the lens optical axis 142). The haptic isolators 105 can extend from an anterior haptic wall 300 to a posterior haptic wall 302.

As shown in this example embodiment, the haptic isolators 105 can be substantially columnar-shaped such that each of the haptic isolators 105 has a substantially circular transverse cross-section. When the haptic isolators 105 are arranged as columns, the haptic isolators 105 can be arranged as a curved colonnade within the haptic lumen 106.

In some embodiments, the haptic isolators 105 can be positioned at fixed intervals along the length of the haptic lumen 106. In other embodiments, a distance separating immediately adjacent haptic isolators 105 can vary along a length of the haptic lumen 106.

Moreover, FIG. 6 illustrates that a width or diameter of one or more of the haptic isolators 105 can be greater or flare out near the ends of the haptic isolator 105 (i.e., closer to the anterior haptic wall 300 and the posterior haptic wall 302) than the rest of the haptic isolator 105.

In other embodiments, the width or diameter of one or more of the haptic isolators 105 can be the same along the entire height or length of the haptic isolator 105.

Although FIG. 6 illustrates the haptic 104 comprising ten haptic isolators 105. It is contemplated by this disclosure that one haptic 104 can comprise between three and up to twenty haptic isolators 105 (or between twenty and thirty haptic isolators 105).

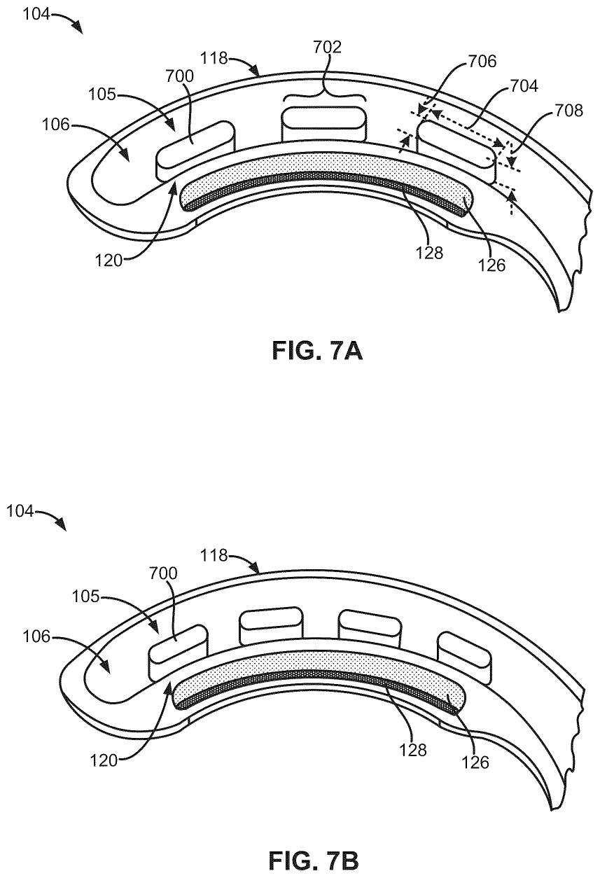

FIGS. 7A and 7B illustrate perspective views of additional embodiments of the haptic 104 comprising a plurality of haptic isolators 105 shaped as isolating blocks 700 or slabs. In FIGS. 7A and 7B, the anterior portion of each of the haptics 104 is not shown for ease of viewing.

In some embodiments, the isolating blocks 700 can have a non-circular transverse cross-section. For example, each of the isolating blocks 700 can have a transverse cross-sectional profile 702 comprising a block length dimension 704 and a block width dimension 706. The block length dimension 704 of the transverse cross-sectional profile 702 can be greater than the block width dimension 706.

In some embodiments, the block length dimension 704 of the transverse cross-sectional profile 702 can be double the block width dimension 706. In other embodiments, the block length dimension 704 of the transverse cross-sectional profile 702 can be more than double (e.g., between 3× to 5×) the block width dimension 706.

Each of the isolating blocks 700 can also have a block height dimension 708. In some embodiments, the block height dimension 708 can be between approximately 1.5 mm to 2.00 mm. For example, the block height dimension 708 can be between approximately 1.75 mm to 1.85 mm (e.g., about 1.84 mm).

In some embodiments, the block width dimension 706 can be between approximately 0.20 mm to 0.60 mm. For example, the block width dimension 706 can be between approximately 0.30 mm to 0.50 mm (e.g., about 0.35 mm).

In other embodiments, the block width dimension 706 can be greater than 0.60 mm depending on the width of the haptic lumen 106. In further embodiments, the block width dimension 706 can be less than 0.20 mm depending on the width of the haptic lumen 106.

In certain embodiments, the block height dimension 708 can be about double the block width dimension 706. In other embodiments, the block height dimension 708 can be more than double the block width dimension 706.

In some embodiments, a ratio of the block height dimension 708 to the block width dimension 706 can be between approximately 3:1 to 10:1. For example, a ratio of the block height dimension 708 to the block width dimension 706 can be between approximately 4:1 to 6:1 (e.g., about 5:1).

In some embodiments, the transverse cross-sectional profile 702 of at least one of the isolating blocks 700 can be substantially obround-shaped (i.e., a rectangle with semi-circular ends). In other embodiments, the transverse cross-sectional profile 702 of at least one of the isolating blocks 700 can be substantially oval-shaped. In further embodiments, the transverse cross-sectional profile 702 of at least one of the isolating blocks 700 can be substantially rectangular or rectangular with rounded corners.

Similar to the haptic isolators 105 shown in FIGS. 1A-1C, 3, 4A-4B, 5B, and 6, the isolating blocks 700 can be configured to counteract or reduce any unintended shape changes caused by laser light 125 directed at the lumen filler 126 and/or the lumen expander 128 in order to fine tune the base power of the IOL 100.

As shown in FIGS. 7A and 7B, the isolating blocks 700 can be positioned within the haptic lumen 106. The isolating blocks 700 can be positioned or located such that none of the lateral sides of the isolating blocks 700 physically contact the radially-outer haptic wall 118 or the radially-inner haptic wall 120. Moreover, as shown in FIGS. 7A and 7B, the isolating blocks 700 can be positioned or located radially closer to the radially-inner haptic wall 120 than the radially-outer haptic wall 118.

Moreover, as depicted in FIGS. 7A and 7B, the plurality of isolating blocks 700 can be arranged in an arc formation or as a curved colonnade within the haptic lumen 106.

In some embodiments, the isolating blocks 700 can be positioned at fixed intervals along the length of the haptic lumen 106. In other embodiments, the isolating blocks 700 can be positioned at variable distances from one another along the length of the haptic lumen 106.

Although FIG. 7A illustrates the haptic 104 comprising three isolating blocks 700 and FIG. 7B illustrates the haptic 104 comprising four isolating blocks 700, it is contemplated by this disclosure that one haptic 104 can comprise between three and up to twenty isolating blocks 700 (or between twenty and thirty isolating blocks 700).

The isolating blocks 700 can be made of the same haptic material used to make the haptic walls. As shown in FIGS. 7A and 7B, the isolating blocks 700 are not made of the composite material used to make the lumen filler 126 and/or the lumen expander 128.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An intraocular lens, comprising:
an optic portion comprising an optic fluid chamber;
a haptic having a proximal end coupled to the optic portion and a distal end, wherein the haptic comprises a haptic lumen extending through at least part of the haptic and in fluid communication with the optic fluid chamber; and
a plurality of haptic isolators disposed within the haptic lumen.

2. The intraocular lens of claim 1, wherein the haptic lumen is surrounded by a radially-outer haptic wall, a radially-inner haptic wall, an anterior haptic wall, and a posterior haptic wall.

3. The intraocular lens of claim 2, and wherein the haptic isolators are configured to limit any radial movement of the radially-outer haptic wall to between 0 and 10 microns in response to laser light directed at the haptic.

4. The intraocular lens of claim 2, wherein the haptic isolators extend from the anterior haptic wall to the posterior haptic wall of the haptic.

5. The intraocular lens of claim 2, wherein each of the haptic isolators comprises lateral sides, and wherein none of the lateral sides of the haptic isolators physically contact the radially-inner haptic wall or the radially-outer haptic wall.

6. The intraocular lens of claim 2, wherein the haptic isolators are positioned radially closer to the radially-inner haptic wall than the radially-outer haptic wall.

7. The intraocular lens of claim 6, wherein a lateral side of at least one of the haptic isolators closest to the radially-inner haptic wall is separated from the radially-inner haptic wall by an inner separation distance, wherein another lateral side of the haptic isolator closest to the radially-outer haptic wall is separated from the radially-outer haptic wall by an outer separation distance, and wherein the outer separation distance is between 1.5× to 3× greater than the inner separation distance.

8. The intraocular lens of claim 1, wherein at least one of the haptic isolators is configured as a column.

9. The intraocular lens of claim 8, wherein the column has a substantially circular transverse cross-section.

10. The intraocular lens of claim 1, wherein at least one of the haptic isolators has a substantially rectangular transverse cross-section, a substantially triangular transverse cross-section, or a substantially oval transverse cross-section.

11. The intraocular lens of claim 1, wherein the plurality of haptic isolators are arranged as a curved colonnade within the haptic lumen.

12. The intraocular lens of claim 1, wherein the plurality of haptic isolators are positioned at fixed intervals along at least a segment of the haptic lumen.

13. The intraocular lens of claim 1, wherein the haptic comprises between three and twenty haptic isolators.

14. The intraocular lens of claim 1, wherein each of the haptic isolators comprises an isolator anterior end, an isolator posterior end, and an isolator segment in between the isolator anterior end and the isolator posterior end, wherein a width or diameter of at least one of the isolator anterior end and the isolator posterior end is greater than the isolator segment in between the isolator anterior end and the isolator posterior end.

15. The intraocular lens of claim 1, wherein a width or diameter of at least one of the haptic isolators remains constant along a length or height of the haptic isolator.

16. The intraocular lens of claim 1, wherein each of the haptic isolators is measurable by an isolator width or diameter and an isolator length or height, and wherein the isolator length or height of at least one of the haptic isolators is more than double the isolator width or diameter.

17. The intraocular lens of claim 1, wherein the haptic isolators are arranged in the shape of an arc, wherein the haptic isolators comprise a distally-most haptic isolator and a proximally-most haptic isolator serving as endpoints of the arc, wherein the arc is measurable by a central angle or arc angle, and wherein the central angle or arc angle is between 70 degrees and 74 degrees.

18. The intraocular lens of claim 1, wherein the haptic comprises at least one of a lumen filler and a lumen expander made of a composite material, wherein the composite material is configured to expand in response to receiving laser light directed at the lumen filler or the lumen expander, and wherein the haptic isolators are not made of the composite material.

19. (canceled)

20. An intraocular lens, comprising:
an optic portion;
a haptic having a proximal end coupled to the optic portion and a distal end, wherein the haptic comprises a haptic lumen extending through at least part of the haptic; and
a plurality of haptic isolators disposed within the haptic lumen in an arc formation.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. An intraocular lens, comprising:
an optic portion;
of 6
a haptic having a proximal end coupled to the optic portion and a distal end, wherein the haptic comprises a haptic lumen extending through at least part of the haptic; and
one or more isolating blocks disposed within the haptic lumen, wherein at least one of the one or more isolating blocks comprises a non-circular transverse cross-section.

40.-51. (canceled)
